(19) United States
(12) Patent Application Publication
(10) Pub. No.: US 2023/0394124 A1
(43) Pub. Date: Dec. 7, 2023

(54) METHOD FOR CONFIGURING DATA ACQUISITION SETTINGS OF A COMPUTING DEVICE

(71) Applicant: KONINKLIJKE PHILIPS N.V., EINDHOVEN (NL)

(72) Inventors: HARM CRONIE, EINDHOVEN (NL); LIEKE GERTRUDA ELISABETH COX, EINDHOVEN (NL); MURTAZA BULUT, EINDHOVEN (NL); VALENTINA LAVEZZO, HEEZE (NL); CORNELIS PETRUS HENDRIKS, EINDHOVEN (NL)

(21) Appl. No.: 18/030,617

(22) PCT Filed: Sep. 26, 2021
(86) PCT No.: PCT/EP2021/076423

§ 371 Date: Apr. 6, 2023

Related U.S. Application Data
(60) Provisional application No. 63/089,179, filed on Oct. 8, 2020.

(51) Int. Cl. G06F 21/32; G16H 10/60; G16H 50/50

(52) U.S. Cl. CPC G06F 21/32; G16H 10/60; G16H 50/50

(57) ABSTRACT

A provided method is for configuring settings of a computing device for providing more efficient and reliable acquisition of data for use in updating a personalized digital model (digital twin) of a subject. The method comprises configuring settings of a biometric authentication function of a computing device so as to provide for overlap in the input data requirements of the biometric authentication function and the input data requirements of the digital twin. There may be different authentication protocols available at the computing device, each requiring different input sensing data. Based on knowledge of these different authentication protocols and data requirements, and based on knowledge of data input needs of a digital twin, an authentication protocol can be selected and/or its settings adjusted, so that when performing biometric authentication, the same acquired sensor data can also be used for deriving physiological parameter data of the subject, for updating the digital twin.

### FIELD OF THE INVENTION

This invention relates to a method for configuring settings of a computing device for improving acquisition of data for use in updating a personalized digital model of a subject.

### BACKGROUND OF THE INVENTION

A recent development in technology is the so-called digital twin concept. In this concept, a digital representation (the digital twin) of a physical system is provided and connected to its physical counterpart, for example through the Internet of things as explained in US 2017/286572 A1. Through this connection, the digital twin typically receives data pertaining to the state of the physical system, such as sensor readings or the like, based on which the digital twin can predict the actual or future status of the physical system, e.g. through simulation, as well as analyze or interpret a status history of the physical twin. It essentially provides a digital replica of the physical object, which permits for example monitoring and testing of the physical object without needing to be in close proximity to it. In case of electromechanical systems, this for example may be used to predict the end-of-life of components of the system, thereby reducing the risk of component failure as timely replacement of the component may be arranged based on its end-of-life as estimated by the digital twin.

Digital twins are most typically used to represent mechanical or electrical devices such as manufacturing machines or even aircraft. Such digital twins are useful to monitor functioning of a device and schedule maintenance for example.

Such digital twin technology is also becoming of interest in the medical field, as it provides an approach to more efficient medical care provision. For example, the digital twin may be built using imaging data of the patient, e.g. a patient suffering from a diagnosed medical condition as captured in the imaging data. Such a digital twin may serve a number of purposes. Firstly, the digital twin rather than the patient may be subjected to a number of virtual tests, e.g. treatment plans, to determine which treatment plan is most likely to be successful to the patient. This therefore reduces the number of tests that physically need to be performed on the actual patient.

The digital twin of the patient for instance further may be used to predict the onset, treatment or development of medical conditions of the patient using a patient-derived digital model, e.g. a digital model that has been derived from medical image data of the patient. In this manner, the medical status of a patient may be monitored without the routine involvement of a medical practitioner, e.g. thus avoiding periodic routine physical checks of the patient. This typically leads to an improvement in the medical care of the patient, as the onset of certain diseases or medical conditions may be predicted with the digital twin, such that the patient can be treated accordingly at an early stage. Moreover, major medical incidents that the patient may be about to suffer may be predicted by the digital twin based on the monitoring of the patient's sensor readings, thereby reducing the risk of such incidents actually occurring. Such prevention avoids the need for the provision of substantial aftercare following such a major medical incident.

A digital twin may be used to simulate a new physical situation or state in a patient using input physical sensor data, for example each time new information or data becomes available. The result is a new output variable field or distribution in a set of output parameters.

In some applications it may be desirable to update a digital twin regularly based for example on sensor data, such that it accurately represents a real physical state of the patient. The input data may include physiological parameter sensor measurements for example. Depending upon the desired output information from the digital twin, there may be different input information requirements. To derive a particular measurement, prediction or parameter from the digital twin there may be a particular set of input information required to be provided to the digital twin.

Updates to model input information may be measured using sensors comprised by a personal device belonging to the patient of whom the digital twin is a replica. This allows the digital twin to be updated regularly even when the patient has been discharged from the hospital and is in the home environment. It is known that the sensor data from sensors of a personal device such as mobile computing device (e.g. smartphone) can be used to derive physiological parameters, such as heart rate, blood oxygen saturation, temperature, breathing rate and many others. However, once in a home environment, the patient can often forget to regularly acquire the physiological parameter measurements, or they may acquire them at times when they are not needed, or acquire them too infrequently. This can result in an incomplete or outdated Digital Twin input.

It would be desirable to find an improved approach to providing regular updated input information to the digital twin.

### SUMMARY OF THE INVENTION

The invention is defined by the claims.

According to examples in accordance with an aspect of the invention, there is provided a computer-implemented method for configuring settings of a biometric authentication function of a computing device based on input data requirements of a personalized digital model of at least a portion of an anatomy of a patient. The method comprises: obtaining a first input indicative of a set of input medical data requirements of the digital model for obtaining, based on running simulations on the digital model, a desired set of output information from the model. The method further comprises obtaining a second input indicative of one or more biometric authentication protocols being executable by the computing device for performing the biometric authentication function on the computing device, wherein each authentication protocol is associated with a set of biometric input data requirements. The method further comprises comparing the input medical data requirements of the digital model with the biometric input data requirements of the one or more authentication protocols. The method further comprises configuring an authentication protocol setting of the computing device based on the digital model input data requirements and based on said comparison.

It is the realization of the inventors that there is overlap between the type of sensor data acquired in performing biometric authentication and the sensing data which can be used to derive the physiological parameters which are used as inputs to personalized digital models (digital twin). Embodiments of the present invention are based on controlling configuration of the authentication settings of a computing device which performs authentication to ensure overlap between the authentication data collected and required input data for a personalized digital model (referred to herein as a ‘digital twin’).

For example, a number of different authentication methods (protocols) may be available for use on the computing device (e.g. fingerprint recognition, iris scan, facial recognition, gait detection), and each may involve acquiring sensor data from a different sensor type (modality) and with different acquisition parameters. Embodiments may comprise, based on the received input data requirements of the digital twin, reconfiguring which authentication method is to be used (by default or on specific future occasions) so that the acquired sensor data, in so doing, can be jointly used also in deriving needed physiological parameter information for supply to the digital twin.

The authentication protocol setting is for example configured so as to achieve a match or sufficient overlap (as defined for example by one or more overlap/matching criteria, rules or algorithms) between the input data requirements of an authentication protocol implemented according to the configured setting and those of the medical input data requirements. In other words, the data that will be acquired in the course of the authentication protocol according to the configured setting will obtain data that is sufficient to meet the digital model input data requirements (at least partly).

In accordance with one or more embodiments, the method may comprise obtaining an indication of a scheduled authentication event comprising scheduled implementation of an authentication protocol, or may comprise obtaining an indication of a default authentication protocol setting of the computing device. The configuring of the authentication protocol setting may comprise altering the scheduled or default authentication protocol, or settings thereof.

Thus, the configuration is done in advance of a future authentication event, where this may be a specific event which is scheduled at a particular time (background authentication), and where for example a user is prompted to perform authentication in accordance with the protocol setting, or is a non-specific future authentication event, being simply the next instance at which authentication of the user is required by the computing device in the course of its normal operations.

In accordance with one or more embodiments, the second input may be indicative of a set of multiple biometric authentication protocols being selectively executable by the computing device, and wherein the configuring the authentication protocol setting comprises selecting one of the authentication protocols.

A comparison may be done between the biometric data requirements of each of the set of protocols and the digital twin data requirements. The method may comprise accessing a datastore or database which stores a list of the different protocols and their input data requirements.

In accordance with one or more embodiments, the first and second inputs may include, or the method may comprise determining, required sensing data to be obtained by the computing device to provide, or to be used in providing, the digital model input data and the biometric data, and wherein the comparing comprises comparing the sensing data requirements.

Here, specifically the sensing data which is needed in order to provide the necessary biometric data and medical data is assessed and compared. Sensing data can include data acquired by physical contact sensors, or non-contact sensing means such as imaging devices, e.g. a camera. The necessary biometric data for a given authentication protocol would then be derived or computed from this raw sensing data, e.g. using one or more dedicated algorithms. For example, the sensing data may comprise image data of the face of a user, and wherein the required biometric data comprises data indicative of facial landmarks for use in facial recognition. Here, the raw imaging data would subsequently be processed to derive the necessary facial landmark data. Likewise, the necessary input medical data for the digital twin may be derived or computed from the raw sensing data. For example, if the sensing data is again image data of a user's face, this can be processed using a dedicated algorithm to derive skin complexion or heart rate information. Thus, there can be overlap between the sensing data requirements of the authentication protocol and the digital twin, even though the actual biometric data and medical data that will be derived from the sensing data are different.

The sensing data requirements may include at least one suitable sensing modality, and optionally may include suitable ranges for one or more sensing modality acquisition parameters.

A modality means the type of sensing device or apparatus which is used, e.g. a fingerprint sensor, a camera, a touch screen etc.

The acquisition parameters refers to adjustable settings of the sensing modality used. For example, a camera may have an adjustable resolution, focus, frame sampling rate, or may be pointed at different areas of the body or may be positioned at different distances from the body. For example, the input biometric data requirements and/or medical data requirements may include a range of acceptable values for different acquisition parameters, e.g. resolution, sampling rate, timing, measurement range, measurement duration etc.

In accordance with one or more embodiments, configuring the authentication protocol setting may include: selecting one of the one or more authentication protocols and/or configuring one or more sensing modality acquisition parameters.

Other options for the authentication protocol setting may include for instance frequency of acquisition of the data (e.g. frequency of repetition of the authentication)

In accordance with one or more embodiments, the authentication protocol setting may be configured so as to achieve at least a partial match between sensing data requirements of an authentication protocol implemented according to the configured setting and those of the medical input data requirements.

For example, the method may comprise selecting an authentication protocol and/or adjusting acquisition parameters in order to achieve the match. Optionally, a minimum matching threshold may be set, wherein complete match is not necessary.

In accordance with one or more embodiments, the configuring the authentication protocol setting may comprise issuing a control instruction to cause the computing device to implement the authentication protocol setting on at least one future authentication event.

This may comprise scheduling one or more future authentication events, or it may comprise setting a default authentication protocol setting for example.

An authentication event means a single instance of implementation of a user authentication. This comprises controlling acquisition of sensing data using one or more sensing arrangements for use as, or in providing, the input biometric data.

In accordance with one or more embodiments, the computing device may be a mobile computing device.

This can be for example a personal computing device such as a smartphone, smartwatch (or other wearable computing device), or a tablet computer.

In accordance with one or more embodiments, the first input may include an indication of one or more sets of predicted future data input requirements of the model at one or more future times, and wherein configuring the authentication protocol setting comprises issuing a control instruction to cause the computing device to schedule implementation of the authentication protocol setting at the one or more relevant future times.

The future data requirements may be predicted by a prediction module, based for example on a current simulated state of the at least portion of the anatomy (generated by the digital model), and potentially based on trends in parameters of the simulated state. For example, if certain clinical indicators are deteriorating, it may mean that a new physiological parameter input will soon be needed, or higher temporal resolution sensing data needed.

In accordance with one or more embodiments, configuring the authentication protocol setting may be further based on input contextual information relating to current or past patient activities, or environmental sensor data. In some examples, authentication protocol settings can be set to best accord with the contextual information, e.g. if a user is driving, a hands-free authentication method may be used, or if a user is walking, an accelerometer-based gait-detection method may be best.

A further aspect of the invention provides a computer program product comprising computer program code, the computer program code being executable on a processor or computer to cause the processor or computer to perform a method in accordance with any example or embodiment outlined above or described below, or in accordance with any claim of this application

A yet further aspect of the invention provides a system for use in configuring settings of a biometric authentication function of a computing device based on input data requirements of a personalized digital model of at least a portion of an anatomy of a patient.

The system comprises: a primary processing arrangement having an input/output for receiving and outputting data. The primary processing arrangement adapted to: receive a first input indicative of a set of input medical data requirements of the digital model for obtaining, based on running simulations on the digital model, a desired set of output information from the model. The primary processing arrangement is further adapted to receive a second input indicative of one or more biometric authentication protocols being executable by the computing device for performing the biometric authentication function on the computing device, wherein each authentication protocol is associated with a set of biometric input data requirements. The primary processing arrangement is further adapted to perform a comparison procedure between the input medical data requirements of the digital model and the biometric input data requirements of the one or more authentication protocols. The primary processing arrangement is further adapted to configure an authentication protocol setting of the computing device based on the digital model input data requirements and based on said comparison.

In accordance with one or more embodiments, the system may further comprise a digital model section, comprising: a data storage arrangement, storing a personalized digital model of at least a portion of an anatomy of the patient. The data storage arrangement is configured to receive one or more model inputs and is configured to simulate an actual physical state of said at least part of the anatomy using the digital model and based on the inputs, to thereby generate one or more model outputs relating to a current or future state of the anatomy.

In accordance with one or more embodiments, the system may include a further processing arrangement for coupling in use to a personalized digital model, and adapted to determine the input data requirements for the digital model based at least in part on a latest set of model outputs from the model, and wherein the primary processing arrangement is arranged to receive the first input from the further processing arrangement.

In accordance with one or more embodiments, the system may include the computing device. The computing device may be a portable or mobile computing device. The primary processing arrangement may be integrated in the portable computing device.

By way of example, the primary processing arrangement may be implemented by the native processing components of the computing device.

There are a wide variety of different options for the architecture of the system and in particular the distribution of the processing and data storage functions, and these will be discussed in greater detail later in this disclosure.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying schematic drawings, in which:

FIG. 1 shows a block diagram of steps of a computer-implemented method according to one or more embodiments;

FIG. 2 illustrates with greater specificity steps of an example of the computer implemented method;

FIG. 3 outlines a possible architecture of components of an example system according to one or more embodiments, including a primary processing arrangement for implementing the computer-implemented method according to one or more embodiments;

FIG. 4 shows with greater specificity the example components of a system as may be provided in accordance with one or more embodiments; and

FIG. 5 shows an outline of an alternative architecture for a system according to one or more embodiments, wherein the primary processing arrangement for implementing the computer-implemented method is integrated in a mobile computing device.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

Embodiments of the invention provide a method for configuring settings of a computing device for providing more efficient and reliable acquisition of data for use in updating a personalized digital model (digital twin) of a subject. The method comprises configuring settings of a biometric authentication function of a computing device so as to provide for overlap in the input data requirements of the biometric authentication function and the input data requirements of the digital twin. There may be different authentication protocols available at the computing device, each requiring different input sensing data. Based on knowledge of these different authentication protocols and data requirements, and based on knowledge of data input needs of a digital twin, an authentication protocol can be selected and/or its settings adjusted, so that when performing biometric authentication, the same acquired sensor data can also be simultaneously used for deriving physiological parameter data of the subject, for updating the digital twin. Thus, the user is spared the need to perform separate medical data acquisition events.

As discussed above, a digital twin (DT) of a patient may require regular updates of the real patient's physiological parameters, such as vital signs, or other model input data, which may be measured using the sensors of personal devices.

The monitoring of vital signs using personal computing devices such as a smartphones and wearables is now a well-known area of technology. It has been applied both for health monitoring, and for biometry. In the field of health monitoring, vital signs such as pulse, heart rate variability, breathing rate, body temperature and blood pressure can be measured using native sensors of the computing device. These can be measured in various circumstances (e.g. in rest, during mild exercise). In the field of biometry, vital signs such as heartbeat and respiration rate may be extracted from physiological sensor signals and enable reliable biometric authentication.

Vital sign sensing, both for health monitoring and for biometry extraction, is enabled in a range of modern personal computing devices (such as smartphones). Example sensing modalities that are used include radar, camera detection, touch sensing and others. Such technology is also suitable for continuous authentication. By way of example, a method for detecting heartbeat using radar is described in the paper: Wu, S, et al. Person-specific heart rate estimation with ultra-wideband radar using convolutional neural networks. 2019, IEEE Access, Vol. 7.

Biometric authentication procedures in such a device may use similar sensor measurements and require regular measurements of physiological parameters which could be conveniently used to update a digital twin model. However, the biometric data acquired for use in authentication may not always meet the data input requirements for properly updating the digital twin model. For example, authentication may be achieved by entering a password, whereas the digital twin may require heart rate data and other physiological parameters measurements as input.

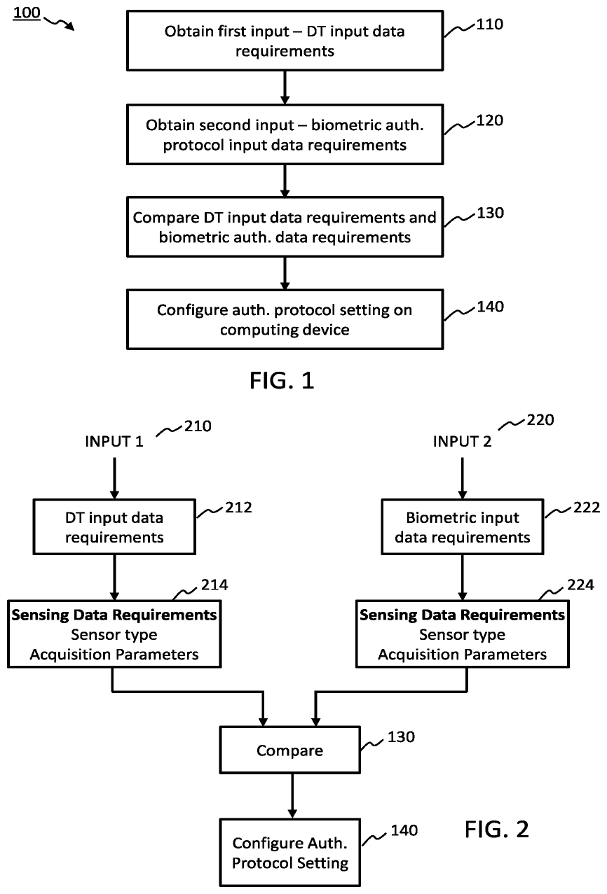

FIG. 1 shows a block diagram of steps of an example computer implemented method 100 according to embodiments of the present invention. The method is for configuring settings of a biometric authentication function of a computing device based on input data requirements of a personalized digital model of at least a portion of an anatomy of a patient.

The method 100 comprises obtaining 110 a first input 210 indicative of a set of input medical data requirements 212 of the digital model for obtaining, based on running simulations on the digital model, a desired set of output information from the model.

The method 100 further comprises obtaining 120 a second input 220 indicative of one or more biometric authentication protocols being executable by the computing device for performing the biometric authentication function on the computing device, wherein each authentication protocol is associated with a set of biometric input data requirements 222.

The method 100 further comprises comparing 130 the input medical data requirements of the digital model with the biometric input data requirements of the one or more authentication protocols.

The method 100 further comprises configuring 140 an authentication protocol setting 340 of the computing device based on the digital model input data requirements and based on said comparison.

Obtaining the first and/or second input may be an active step of acquiring the input, or may be a passive step of receiving the input. The first input may for example be obtained from a datastore recording input data requirements or for example from a processing or storage component of a digital twin sub-system. Further description will follow later.

In some examples, obtaining the second input may comprise communicating with a local or remote datastore storing a database which records a set of different authentication protocols and their corresponding biometric data input requirements. For example, the second input may be indicative of a set of multiple biometric authentication protocols being selectively executable by the computing device, and wherein the configuring the authentication protocol setting comprises selecting one of the authentication protocols.

The configuring 140 an authentication protocol setting may comprise for example generating a control output to cause the computing device to implement the authentication protocol setting for at least one future authentication event.

FIG. 2 schematically illustrates with greater specificity the steps of an example of the computer implemented method 100. The first 210 and second 220 inputs are received or obtained. The first input includes the digital model input data requirements 212. The second input includes the biometric input data requirements 222.

In preferred examples, each of the first 210 and second 220 inputs include, or the method comprises determining, required sensing data 214, 224 to be obtained by the computing device to provide, or to be used in providing, the digital model input data and the biometric data. The comparing 130 of the data input requirements 214, 224 in this example comprises comparing the sensing data requirements 214, 224.

The sensing data requirements means the raw sensing data requirements. In practice the acquired sensing data, during execution of an authentication event, may be further processed in order to derive from the sensing data the necessary biometric and medical data. This subsequent processing can be done as part of the method of the invention, or separately to it. For example, the sensing data may comprise image data of the face of a user, and wherein the biometric data comprises data indicative of facial landmarks for use in facial recognition authentication. Here, the raw imaging data would subsequently be processed to derive the necessary facial landmark data. Likewise, the necessary medical data for the digital twin may be derived or computed from the raw sensing data. For example, if the sensing data is again image data of a user's face, this can be processed using a dedicated algorithm to derive skin complexion and/or heart rate information. Thus, there can be overlap between the sensing data requirements of the authentication protocol and the digital twin medical data requirements, even though the actual biometric data and medical data that will be derived from the sensing data are different.

The sensing data requirements 214, 224 for each of the digital twin and the biometric authentication may include at least one suitable sensing modality, and preferably may further include suitable ranges for one or more sensing modality acquisition parameters.

A modality means the type of sensing device or apparatus which is used, e.g. a fingerprint sensor, a camera, a touch screen, an audio sensor, an accelerometer.

The acquisition parameters refers to adjustable settings of the sensing modality used. For example, a camera may have an adjustable resolution, focus, frame sampling rate, or may be pointed at different areas of the body or may be positioned at different distances from the body. For example, the input biometric data requirements and/or medical data requirements may include a range of acceptable values for different acquisition parameters, e.g. resolution, sampling rate, timing, measurement range etc.

In this set of examples, configuring 140 the authentication protocol setting may include selecting one of the one or more authentication protocols and/or configuring one or more sensing modality acquisition parameters.

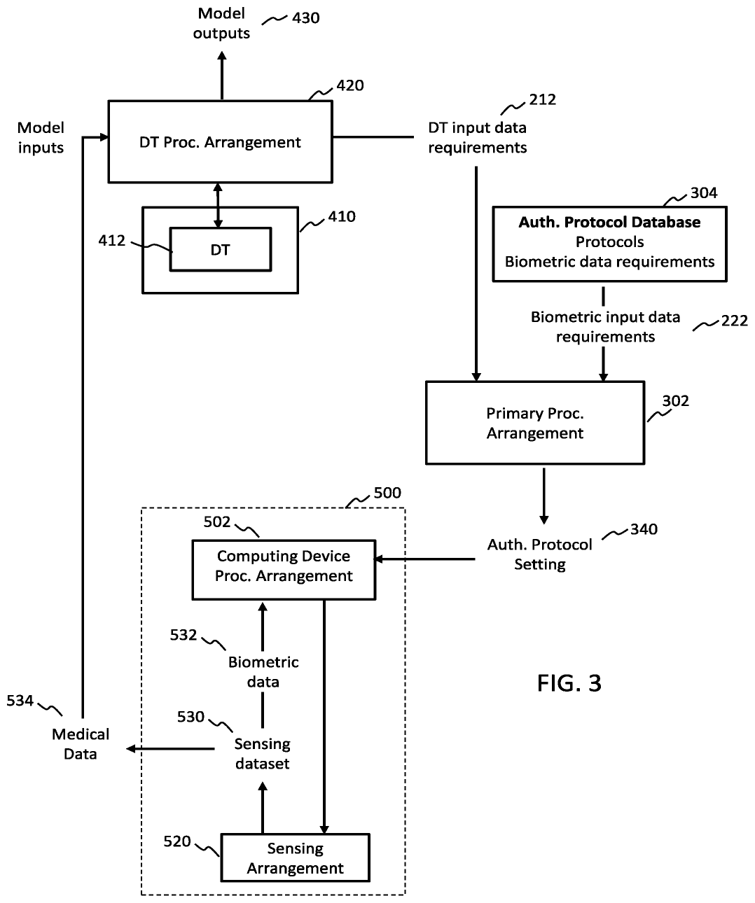

To further explain the method, reference will now be made to FIG. 3 which outlines the architecture of an example system as may be provided in accordance with one or more embodiments. The system includes a primary processing arrangement 302 which may be provided and configured to implement the computer-implemented method 100 in accordance with any of the examples outlined above or described below, or in accordance with any claim of this application.

According to one aspect of the invention, just the primary processing arrangement 302 may be provided, the primary processing arrangement including an input/output or communication module configured to facilitate communication with other components of the illustrated system, as required. In a further aspect of the invention, a system may be provided, wherein the system includes the primary processing arrangement, in addition to any one or more of the other system components outlined in FIG. 3. There are different options for the architecture of the system, as will be explained in more detail later.

As illustrated in FIG. 3, the primary processing arrangement 302 is in operative communication with a Digital Twin (DT) sub-system or Digital Twin section, which comprises a Digital Twin (DT) processing arrangement 420, which is arranged to provide the digital twin input data requirements 212 as an input to the primary processing arrangement 120. The DT processing arrangement 420 is operatively coupled with a data storage arrangement 410 which stores a personalized digital model 412 of at least part of an anatomy of a patient. The personalized digital model 412 may be otherwise referred to in this disclosure as a digital twin.

The digital model 412 is configured for simulating a state of at least a portion of the anatomy of the subject based on adjustment of a set of one or more model input parameters. The model is operable to provide output information 430 related to the simulated state of the anatomy, for example one or more output parameters. These may correspond to physiological or anatomical parameters of the patient. The digital twin may simulate a digital representation of a physical state of a portion of the subject's anatomy and/or may simulate one or more properties of a broader health or physiological state of the patient. Aspects of the patient physiology which may be modelled by the Digital Twin include, for example, 3D geometry (e.g. of the bones/organs/tissue/veins), motion or biomechanics, flow dynamics (e.g. blood, air, heat), organ function (e.g. renal output, cardiac contraction), and/or disease progression.

The digital twin 412 way in some examples integrate artificial intelligence, machine learning and/or software analytics with spatial network graphs to create a ‘living’ or live digital simulation model of the at least portion of the patient's anatomy. Input data, such as physiological sensor data, may be used to update and change the digital twin dynamically, and optionally in real time, such that any changes to the patient as highlighted by the data are reflected in the digital twin. In some examples, the digital twin may thus form a learning system that learns from itself using the sensor data. The digital twin is thus preferably a dynamic model which dynamically develops or updates so as to provide an accurate representation of the patient's real anatomy.

The digital model 412, i.e. the digital twin, of the patient may be initially developed from patient data, e.g. imaging data such as CT images, MRI images, ultrasound images, and so on. For example a medical scan may be conducted of the patient, and/or a set of one or more physiological or anatomical parameter measurements taken for the patient, and the digital model constructed based on this.

A typical workflow for creating and validating a 3D, subject-specific biophysical model is depicted in “Current progress in patient-specific modeling”, by Neal and Kerckhoff, 1, 2009, Vol. 2, pp. 111-126. For example, in case of a digital twin representing part of the cardiovascular system of the patient, such a biophysical model may be derived from one or more angiograms of the patient.

In operation, the processing arrangement 420 may develop or update the digital twin using received medical sensing data in order to simulate the actual physical state of the at least portion of the anatomy of the patient.

Development and implementation of digital twin models for various example applications are described in the literature for this field. By way of example, implementation details for various example digital twin models are described in the following papers: Gonzalez, D., Cueto, E. & Chinesta, F. Ann Biomed Eng (2016) 44: 35; Ritesh R. Rama & Sebastian Skatulla, Towards real-time cardiac mechanics modelling with patient-specific heart anatomies, Computer Methods in Applied Mechanics and Engineering (2018) 328; 47-74; Hoekstra, A, et al, Virtual physiological human 2016: translating the virtual physiological human to the clinic, interface Focus 8: 20170067; and “Current progress in patient-specific modeling”, by Neal and Kerckhoff, 1, 2009, Vol. 2, pp. 111-126.

Details are also outlined in “Computational Biomechanics for Medicine”, Grand R. Joldes et al, Springer.

In general, the digital model, e.g. of an organ or tissue area of the patient, incorporates a number of different (e.g. heterogeneous) material properties as parameters of the model, which may include blood vessels, muscles, fat, lining tissue, bones, calcified areas, which each have specific (biomechanical) material properties. These material properties form parameters for the model to allow physical development of the anatomy with changing physiological circumstances to be modelled.

The model simulates the real physical state of the patient. By feeding the model appropriate input information, the model is able to provide computed output information relating to one or more physiological or anatomical parameters. This may be based on running certain simulations on the updated model by tuning input parameters of the model, or may be based on using one or more algorithms encoded in the model, based on input information, to compute or derive physiological information about the state of the patient's anatomy.

From an up-to-date digital twin, one or more physiological or anatomical parameters of the modelled anatomy (output information) can thus be extracted or read off from the model. These may advantageously be parameters which are not directly measurable using sensors in real time, so that the model provides an insight into physical parameters beyond those that can be measured using standard sensors or imaging equipment.

The DT processing arrangement 420 is configured inter alia to determine input data requirements for the digital twin. The input data requirements may be determined based on a particular aim or goal, for example, for updating parameters of the digital model 412 in order to update the simulated state of the at least portion of the subject anatomy, or to provide a prediction or estimation of a current or future state of a particular property of the subject's health or physiology.

Also schematically illustrated in FIG. 3 are a subset of components of an example computing device 500. The computing device may be a personal computing device. It may be a mobile computing device, such as a smartphone, a tablet computer or a wearable computing device such as a smartwatch or fitness band. However, it may in other examples be any other kind of computing device, e.g. a tabletop computing device, or a home appliance with authentication function which utilizes physical sensors, e.g. a television, a door entry system, or a smart home system.

The computing device 500 includes a sensing arrangement 520 which comprises one or more sensing components. The sensing arrangement is operatively coupled with a processing arrangement 502 of the computing device, which may comprise one or more processors or integrated circuits. The computing device processing arrangement 502 is adapted in operation to control the sensing arrangement to acquire a sensing dataset 530 for use in biometric authentication. Further processing may be applied to the sensing dataset to derive data relevant for biometric authentication (biometric data 532), and wherein the computing device processing arrangement 502 is adapted to compare the derived biometric data with a stored database of biometric profiles, the database storing pre-acquired biometric data or signatures for one or more authorized users.

The one or more sensing components comprised by the sensing arrangement 520 may be native sensing components of the computing device, which are utilized during an authentication event to acquire a sensing dataset 530 which is suitable for deriving needed biometric data. Although FIG. 3 shows the sensing arrangement 520 as a discrete component, it may represent a distributed set of sensing components, comprised by a plurality of different units or modules of the computing device.

The sensing dataset 530 acquired using the sensing arrangement 520 may also be further processed to derive medical data 534, for example data representative of one or more physiological parameters. This processing may be done by the computing device 500, or may be done externally to the computing device by a further system or component with which the computing device is communicatively coupled. In either case, the processing of the sensing data 530 to derive the medical data 534 is based on the input medical data requirements 212 of the digital twin 412, and the derived medical data includes data which matches at least a subset of the DT input data requirements 212.

In operation, the derived medical data 534 is communicated to the digital twin (DT) processing arrangement 420, for use in updating the personal digital model 412. In some examples, the raw sensing dataset 530 may be communicated to the DT processing arrangement 420 and wherein the DT processing arrangement 420 is adapted to derive the medical data 534.

The sensing arrangement 520 of the computing device may comprise sensing components or elements of any of a wide range of different sensing modalities. One non-limiting example includes radar sensing. For example, a radar sensing module may direct radio waves toward a user's chest area, and sense the reflections therefrom. These can be used to determine a variety of parameters, including pulse, heart rate variability and characteristics of movement of the user's chest, such as speed or velocity of chest motion (caused by the movement of the heart). Characteristics patterns in one or more of these properties can also be used for biometric authentication in some examples.

A further non-limiting example includes use of a camera for acquiring image data. For example, an RGB color video of a user's face may be acquired. By applying dedicated algorithms, the image data from such a video can be used to determine a variety of parameters including pulse, breathing rate, and an estimation of skin temperature. In some examples, an RBG-D camera may be used, which is able to acquire additional depth information (RGB-D). This can assist in more accurately determining the physiological parameters. The camera image data can be used to detect further parameters such as a characteristic movement of a user's hand when interacting with a screen, or facial recognition based on shape and position of facial features.

A further non-limiting example includes use of a motion sensor, such as an accelerometer. The motion data can be used to detect gait movement patterns of the user, which information may be used for authentication, based on pre-stored information about characteristics gait patterns of authorized users.

A further non-limiting example includes use of a touch screen. This can be used to acquire, for example, fingerprint sensing data, and potentially simultaneously acquire physiological parameter data such as pulse or blood oxygen saturation. The touch screen may also acquire information such as a characteristic movement of the user's hand when interacting with the touch screen. This can be used for authentication.

A further non-limiting example includes use of audio sensing elements, such as microphones. These can be used to detect properties or features of speech. This can be used for authentication, based on pre-stored speech pattern information for authorized users. The speech pattern information can also be used to determine properties of a user's emotional state, which can be helpful in assessing a person's mental health state.

A further non-limiting example includes use of iris scanning. This can be done with a camera. This provides biometric data, but also can be used to derive health-related data based on retinal image analysis which be used to determine diabetic retinopathy progression or macular degeneration progression.

In operation, the primary processing arrangement 302 is adapted to obtain input medical data requirements 212 from the DT processing arrangement 420, and adapted to obtain biometric input data requirements 222. In the example illustrated in FIG. 3, the biometric input data requirements 222 are obtained by communicating with an authentication protocol database 304 which stores a record of a plurality of biometric authentication protocols which are selectively executable by the computing device 500 for user authentication. The database stores a record of the biometric data requirements associated with each of the different authentication protocols. The processing arrangement 302 may receive from the protocol database 304 a data output indicative of the set of authentication protocols and the biometric data requirements 222, or it may communicate with the authentication protocol database to retrieve or access the list of protocols and data requirements.

The primary processing arrangement 302 also receives the medical input data requirements 212 from the DT processing arrangement. These are compared with the biometric data requirements 22 and, based on the comparison, the primary processing arrangement generates a control output 340 for configuring an authentication protocol setting of the computing device 500. This output is communicated for example to the computing device processing arrangement 502.

As discussed above, an authentication protocol refers to an algorithmic process which is for execution by the computing device 500, and which comprises acquiring a certain set of sensing data 530 using a defined set of sensing modalities, and preferably with a defined set of acquisition settings or parameters, and processing that data in a certain way to derive a set of biometric data which can be used for authentication purposes. The computing device may store locally a plurality of different authentication protocols which it is operable to implement, and wherein the authentication protocol database 304 mirrors the protocols stored on the computing device, or wherein the authentication protocol database 304 shown in FIG. 3 is stored on the computing device itself.

The authentication protocol setting 340 output by the primary processing arrangement 302 may include a selection of one of the one or more authentication protocols from the database 304 and/or it may comprise a configuration for one or more acquisition parameters of the sensors 520 to be used in acquiring the biometric data. As discussed above the authentication protocol can include sensing data requirements which specify a set of one or more sensing modalities to be used, and optionally an acceptable range for one or more acquisition parameters. The acquisition parameters correspond to adjustable settings of the sensing modality used. For example, a camera may have an adjustable resolution, focus, frame sampling rate, or may be pointed at different areas of the body or may be positioned at different distances from the body.

In configuring the authentication protocol setting 340, the primary processing arrangement is adapted to issue a control instruction to cause the computing device 500 to implement the authentication protocol setting on at least one future authentication event.

This may comprise scheduling one or more future authentication events, or it may comprise setting a default authentication protocol setting for example. An authentication event means a single instance of implementation of a user authentication. This comprises controlling acquisition of sensing data using one or more sensing arrangements for use as, or in providing, the input biometric data.

At least one future authentication event means for example the next authentication event, all future authentication events, or one or more specific scheduled future authentication events. The computing device may comprise an authentication scheduling module which schedules future authentication events at specific future times and according to specific authentication protocols, and wherein the control instruction 340 comprises adjusting the schedule. In some examples, the processing arrangement may be adapted to interrogate the schedule in advance of determining the authentication protocol setting 340, and to compare the biometric data requirements of one or more future scheduled authentication events with the DT medical data requirements 212, and make adjustments to improve overlap or match between the two. In some examples, the processing arrangement 302 may be adapted to interrogate the computing device to identify a default authentication protocol setting, to compare the biometric data requirements of the default setting with the DT input data requirements 212 and perform adjustment of the default setting on the computing device to improve overlap or match between the two.

In either case, if the scheduled authentication events, or the default authentication protocol setting, will not result in a gathered sensing dataset 530 which also meets the input medical data requirements 212, the primary processing arrangement 302 is adapted to adjust the scheduled authentication events or the default authentication setting to improve the correspondence. Adjustments which can be made include, for example: the frequency of measurements taken, the sample rate of a measurement; the duration of a measurement (sensor signal collected over a certain period of time); the type of sensor (sensing modality) used to make the measurement; the range and/or resolution of the variables to be measured.

In the case of interrogating the schedule of future authentication events, this may comprise an analysis of the authentication protocol of a next scheduled authentication event, or may comprise an analysis of the protocols for authentication events over an extended time period, e.g. a whole day. The DT input data requirements 212 may for example specify the input data requirements for the DT over a certain time period, e.g. a specified list of input data is needed to be collected over x period of time. In the latter case, the primary processing arrangement may be adapted to compare the biometric data requirements of the full set of scheduled events over the time period x with the DT input data requirements 212 over period x. The primary processing arrangement 302 may then adjust the schedule of authentication events in order to improve the overlap between the two. This may comprise changing the authentication protocols to be deployed at scheduled times, adding new authentication events, or adjusting data acquisition settings associated with each authentication event.

A schedule of authentication events at specific times may be used of example for computing devices in environments where data security and authentication is important, and where the computing device is used regularly over an extended period of time. Examples include for instance personal computing devices of medical personnel used in the course of their duties and which may provide access to confidential patient information. Here, the computing device may be configured to regularly re-prompt the user for authentication data, to confirm the authorization of the user. Thus an authentication schedule may be used.

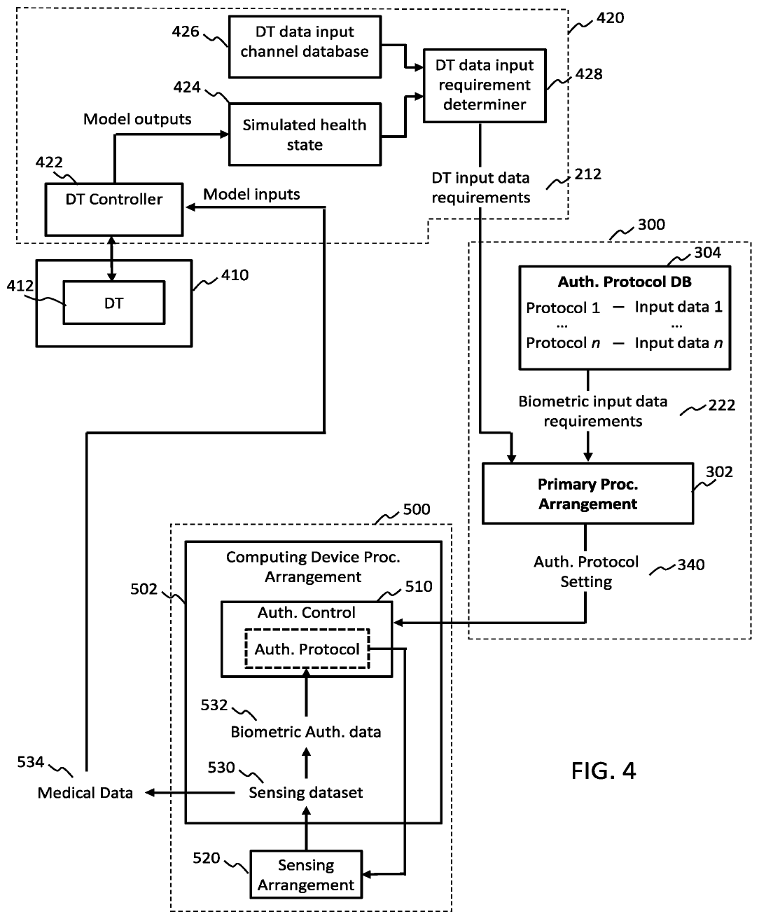

FIG. 4 outlines with greater specificity components of an example system according to one or more embodiments. Any one or more of the additional components shown in FIG. 4 compared to FIG. 3 may be advantageously incorporated in the system of FIG. 3 in accordance with various embodiments.

FIG. 4 shows the digital twin processing arrangement 420 which includes a plurality of processing and storage modules. Although each module is shown as a discrete component, this is not essential and the functionality of each module may be performed by one or more components, or the functionality of all of the modules may be performed by a single processing component in some examples.

The digital twin processing arrangement 420 comprises a controller (“DT controller”) 422 which communicates with the data storage arrangement 410 which stores the personalized digital model of the at least portion of the subject's anatomy (the digital twin) 412. The DT controller 422 is arranged to receive from the computing device 500 the medical data 534 and to communicate with the stored digital twin model 412 to update the model based on the medical data inputs. The DT controller may receive other medical data inputs in addition to those provided by the computing device 500.

The DT processing arrangement 420 further includes a storage module 424 which stores a representation of a simulated state of the subject, or a portion of the anatomy of the subject. This may comprise a set of simulated variables having values which represent the user's real world state at a given moment. The simulated health state may be a recent, current or future state and is created when running the digital twin model 412.

The DT processing arrangement 420 further includes a digital twin (DT) data input channel database 426. This stores a record of all of the input medical data variables (data input channels) which the digital twin model is able to receive. The input channel database may be populated with a record of various types of information that are needed in order to run different simulations or to determine different parameters, e.g. medical images, medical test results, physiological measurements.

The DT processing arrangement 420 further includes a DT data input requirement determiner 428. This may be a processing component programmed with one or more algorithms operable to determine the input data requirements of the digital twin model 412, based on the input data channels 426 of the DT and optionally based on the stored current or future health state 424. For example, the requirement determiner may take as an input the data entries in the DT data input channel database 426 and the simulated health state 424 and generate an output indicative of a set of input health data requirements 212. The input health data requirements may comprise a list or description of required input data for the digital twin model 412. These may include a set of sensing data requirements, where this may include sensing modalities to be used and/or data acquisition parameters (such as measurement range, resolution, timing, amount of data, order of collection of variables, body location of measurement). Additionally or alternatively, it may comprise a list of one or more physiological variables (e.g. pulse, breathing rate, etc.).

Optionally, the DT input data requirement determiner 428 may additionally take as an input contextual information regarding the patient's activities, e.g. derived from sensor measurements acquired by the computing device 500, or from an electronic agenda or diary of the patient, or from one or more user inputs. Optionally, the DT input data requirement determiner 428 may additionally take as an input environmental information such as environmental temperature, humidity or air quality. These may for example be derived from sensors, from a database or received as a user input.

DT data input requirement determiner may be configured to perform a step of determining if the current simulated health state fulfils a certain one or more criteria, the criteria defining requirements for meeting or more goals or aims (e.g. simulating a certain anatomical region or a certain set of one or more health parameters, or keeping the simulated state updated with a certain regularity). If the criteria are not met, the DT data input requirement determiner may determine what additional input information is needed in order for the criteria to be met.

By way of one example, the DT data input determiner may determine if new input data is needed for the digital twin model 412 based one or more of the following factors: based on evaluating the date or time at which the most recent input information used to create the simulated health state 424 was obtained; based on determining a measure of the accuracy or uncertainty of the simulated health state; based on detected change in environmental data; based on a change in the simulated health state (e.g. deterioration); based on evaluating a change in simulated health state 424 after running a test simulation on the digital twin model 422 with an artificially generated set of new input health data, e.g. randomly generated or generated using an estimation or extrapolation algorithm; based on an input from an external source, e.g. a user input requesting a particular parameter to be simulated.

If the DT data input requirement determiner determines that new input data is required for the digital twin model 412, it performs a step of determining the new input data requirements 212 based on the entries in the DT data input channel database 426 (which lists all of the possible data inputs which the digital twin model is able to receive), and based on the simulated health state 424, and based on the evaluation outlined in the preceding paragraph.

The requirement determiner 428 generates an output indicative of the determined DT input data requirements 428. This may include an indication of the sensing data requirements (which sensor modalities), and an indication of acceptable ranges for one or more acquisition parameters such as: a required resolution or precision of the measurement, and/or timing parameters of the measurements, e.g. regularity of measurement acquisition.

Based on received input data 534, the digital twin model 412 is operable to calculate a desired simulated health state 424. For example, the DT controller 422 may configure parameters or settings of the model to induce it to model a particular anatomical area or feature, or a particular one or more health or physiological or anatomical parameters. The digital model 412 is configured to generate the required simulation based on the received input medical data.

By way of example, the digital twin model 412 which is used, and the simulated health state 424 which is obtained, could take one or more of the following forms.

In one example, the DT model 412 may be a 3D model of the physical anatomy of the heart including morphology, cardiac muscle activity and a position of various structural components of the heart, e.g. movements of the heart valves, size of the ventricles and atria. The model may be patient-specific and can be constructed based on medical imaging scans of the relevant area of the patient anatomy, as discussed above. Alternatively, a generic model may be used. The model could be used to generate simulations of health states such as a general simulation of overall heart health, and estimated risk of a heart attack.

According to a further example, the DT model 412 may be a blood flow model of at least a region of the user's skin (e.g. hands or face), and related thermal bio-regulation information. This may be based on a fluid dynamic model of blood flow as well as a physical model of the structure of the blood vessels through the skin region. In this example, the model could be used to generate simulations of health states such as likelihood of a heat stroke.

According to a further example, the DT model 412 may be a biomechanical model of a range of motion and muscle tone of particular limbs, e.g. the hands or legs of the user. In this example, the model could be used to generate simulations of health states such a current physical state of the limb, or of particular parameters linked to progression of the health state in the future such as predicted rate of recovery after an injury or surgery, or progression of one or more pathologies such as neuromuscular diseases such as Parkinson's and MS.

FIG. 4 further shows a primary processing system 300 comprising the primary processing arrangement 302, as discussed above, and further comprising the authentication protocol database 304 as discussed above. The primary processing system may be provided in accordance with an aspect of the invention, or just the primary processing arrangement 302 may be provided.

FIG. 4 further shows a subset of components of an example computing device 500. The computing device processing arrangement 502 (discussed above and shown in FIG. 3) is shown comprising an authentication control module 510. This is adapted to store a record of the particular authentication protocol which is to be executed. In operation, the authentication control module is adapted to receive a control instruction 340, generated by the primary processing arrangement 302, indicative of a requested configuration of an authentication protocol setting. The authentication control module 510 adjusts the relevant protocol setting responsive to this instruction. This may comprise adjusting a schedule of future planned authentication events, or may comprise adjusting a default authentication protocol setting for authentication events. The authentication control module 510 is adapted to control execution of authentication events in accordance with the authentication protocol configurations communicated by the primary processing arrangement 302. This comprises controlling a relevant set of sensing components comprised by the sensing arrangement 520 to acquire sensor data 530. The computing device processing arrangement 502 and/or the authorization module 510 may process the sensing data 530 to derive biometric data 532, and then interrogate a local or remote database of biometric signatures of authorized users to determine a result of the authorization event (e.g. user authorized or not, and/or level of authorization).

The sensing dataset 530 is also output from the computing device processing arrangement 502 to provide the medical dataset 534 for use in updating the digital twin model 412. In some examples, the raw sensing data is output by the processing arrangement, and for example communicated to the DT controller 422. The DT controller may apply further processing to derive the required DT input data parameters (as determined earlier by the DT data input requirement determiner 428). The DT controller 422 may then communicate with the DT model 412 stored on the storage arrangement 410 to update the model with the derived input data. Alternatively, the processing of the derived sensor dataset 530 to derive the relevant input medical data for updating the DT model 412 may be done elsewhere, for example by the processing arrangement 502 of the computing device 500 itself, or by a further remote processing arrangement, for example a remote server, e.g. a cloud-based processing arrangement.

The example system architecture shown in FIG. 3 and FIG. 4 represents one set of examples. Although the basic components and data flow may be common among all embodiments, there are a wide variety of different options for the particular architecture of the system and, in particular, the distribution of the processing and data storage functions.

In the example of FIG. 3 and FIG. 4, the primary processing arrangement 302 is shown as a discrete processing component. However, this is not essential and its processing functionality may instead be distributed among a plurality of processing components comprised by one or more separate units. In some examples, the primary processing arrangement may be comprised by a dedicated processing unit. This may be a dedicated computing device. In some examples, it may be a cloud-based processing arrangement, and wherein the digital twin subsystem and the computing device 500 communicate with the processing arrangement though a remote communication channel, such as an Internet connection.

In accordance with one or more further embodiments, the system may include the computing device 500. The computing device may be a portable or mobile computing device. The primary processing arrangement 302 may be integrated in the portable computing device in some examples.

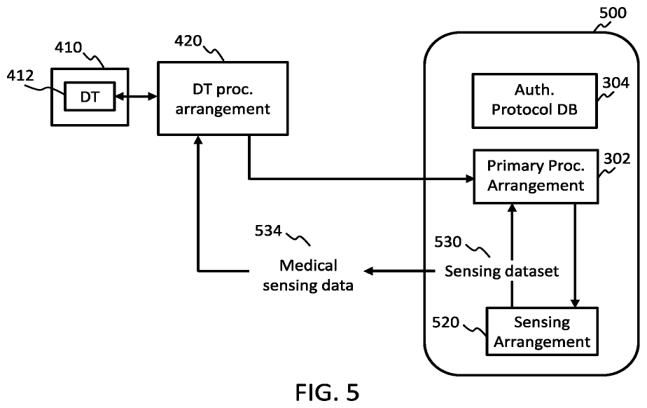

FIG. 5 schematically outlines an example of such a system architecture.

In this example, the primary processing arrangement 302 is implemented by the native processing components of the computing device. In this example, the authentication protocol database 304 is also stored on a local data storage component of the computing device 500. In further examples however, the authentication protocol database 304 may be stored in a further remote datastore, and the primary processing arrangement 302 is adapted to communicate with the remote datastore to access the database. Communication may be via an internet link for example.

In some examples, the personalized digital model 412 (digital twin) may also be stored on the computing device 500, or the processing arrangement 420 which controls execution of simulations using the digital twin may be implemented by the computing device processors. The digital model itself may be stored elsewhere, for example in a remote datastore or in the cloud.

In further examples, the primary processing arrangement may be located elsewhere, for example in a dedicated computing device, or in the cloud and is a distributed processing arrangement, or it may be implemented by a system storing and operating the digital twin, e.g. a hospital computing system. In some examples, the portable computing device includes a client app communicable with the primary processing arrangement via a communication channel (e.g. Web link, or Wi-Fi, or LAN or Bluetooth).

To further illustrate embodiments of the invention, a number of example application cases will now be outlined.

By way of one example case, a patient may have an appointment scheduled with their doctor a certain time in the future, e.g. in 3 weeks. The patient does not have access to any wearable computing devices (e.g. fitness tracker or smartwatch) to monitor their health. It is clinically useful for the doctor to be provided some information relating to the patient health over the period leading up to the appointment, e.g. vital sign data such as heart rate.

As such, the DT processing arrangement 420 may generate an output indicative of the clinically desired input medical data requirements of a personalized digital model 412 of the patient. The primary processing arrangement may then communicate with the computing device processing arrangement 502 to adjust the schedule of authentication events over the period leading up to the visit, such that the authentication events use authentication protocols and/or settings which collect sensing data which can also be used to derive heart rate information. For example, authentication protocols may be chosen that are based on fingerprint or facial recognition, in such a way that also heart rate measurements can be obtained. In addition, if activity tracking is enabled on the mobile phone of the patient, this information can be used to contextualize the heart rate measurements (e.g. heart rate in rest versus heart rate after a long walk).

Instead of changing a schedule of authentication events, the primary processing arrangement 302 may instead simply change the default authentication protocol setting so that all authentication events over the period leading up to the doctor appointment collect sensing data which can be used to derive heart rate data.

By way of a further example case, a patient with high blood pressure may have an annual check-up scheduled with his or her doctor a certain period in the future, e.g. two weeks. The patient has been previously diagnosed with high blood pressure. The patient has been monitoring his or her own health by taking regular blood pressure measurements at home, and wearing a fitness tracker which measures his or her activity and heart rate. These data are used as input to a personalized digital twin 412 for the patient. The digital twin includes a digital model of the patient's heart health and is adapted to generate an output 430 indicative of predicted risk of cardiovascular illness or disease in the future.

By way of an example case, the digital twin output predictions 430 (e.g. a simulated heath state 424) indicate that a condition of the patient's heart may be deteriorating. Based on this, the digital twin processing arrangement 420 adjusts the input medical data requirements 212, so as to enable more accurate assessment of the risk of further deterioration of cardiovascular condition. The changes to the DT input medical data requirements 212 may call for additional information regarding whether the patient is experiencing certain symptoms and whether he or she has been taking the medication as prescribed. In response, the primary processing arrangement 302 may be adapted to adjust the authentication protocol setting of the computing device 500 so that the default authentication protocol setting involves use of speech recognition. During authentication, one or more questions may be presented on a display of the computing device asking for the required information about symptoms and medication adherence. The verbal answers from the patient can be processed with speech recognition algorithms to discern the cognitive content of the answers, which information can be provided to the digital twin processing arrangement 420, and the audio recordings can also be processed to derive speech pattern information, which can be used as biometric data 532 for the biometric authentication function.

In the above example use case, context may also be taken into account when scheduling the authentication. For example, if the computing device performs scheduled authentication events, the events may be scheduled at times outside of the patient's working hours, such that the verbally provided answers to the health-related questions may remain private. Additionally, the patient may be provided the option to choose an alternative authentication protocol.

By way of a further example case, a patient may have a digital twin model which models his or her vascular network, for simulating vascular response to various environmental and medical inputs.

The digital twin model may simulate the user-specific vascular response to stimuli which can be used for example for user-specific heat stress management or topical drug delivery. Such a model may be generated by correlating the behavior of the vascular network (e.g. in the form of measured vasodilation, as detected from the body via Near-Infrared (NIR) imaging housed in a computing device such as a mobile phone) in response to various variables such as environmental temperature, user exertion, medication and user-specific medical factors.

The performance and accuracy of the model will improve with increasing volume and range of input data provided to the model. In one example case, the model may be lacking input vascular response data for higher environmental temperature conditions (e.g. >23° C.). Therefore, the digital twin processing arrangement 420 determines that input data 212 is required comprising an NIR image of the patient vasculature under the relevant environmental condition. The primary processing arrangement 302 may communicate with the computing device processing arrangement 502 to cause adjustment of the authentication event schedule, such that, when the specified environmental condition is satisfied (e.g. as measured by local temperature sensors, or based on data from an Internet source), an authentication protocol is adopted for a next authentication event which involves acquisition of sensing data suitable for deriving the vascular response information. For example, an authentication protocol may be used in which image data of a vasculature structure of a portion of the patient's body (in particular, the portion of the body used as the basis for the vasculature model, e.g. the back of the right hand) serves as biometric authentication information 532. The same acquired image data can also be used to determine the patient vasodilation response.

Similarly, in a further example, the authentication schedule of the computing device could be adjusted to cause it to utilize a certain authentication protocol dependent on the patient being in a particular geolocation (e.g. a geolocation corresponding to a particular altitude). Here, GPS data acquired by the computing device may be used to determine when the geolocation condition is met.

As discussed above, in accordance with one or more embodiments, the primary processing arrangement and/or the computing device may be adapted to acquire environmental or contextual information. The authentication protocol setting 340 configuration by the primary processing arrangement 302 may depend in part on the environmental or contextual information. The authentication protocol setting may include conditionality constraints pertaining to the use of particular authentication protocols or settings by the computing device. The conditionality constraints may relate to contextual and/or environmental information. For example, if a user is driving, a hands-free authentication method may be used, or if a user is walking, an accelerometer-based gait-detection method may be used. In some example, the conditionality constraints may require that the authentication events are always executed when the patient is in a particular reference state, e.g. stress level, as determined by a sensor of the computing device or of a further device.

In accordance with one or more embodiments, contextual or environmental information may further be derived concurrently with execution of authentication events, so that the derived medical information can be labelled or tagged according to the context of the measurement acquisition. For example, it is clinically useful to know a particular mental or physical state of a patient when measurements are taken. In particular, a stress level of the patient may be important. For example, an acquired physiological parameter measurement, derived from the acquired sensing dataset 530, may be labelled with a stress level obtained from for example a skin impedance sensor in a further device such as a smartwatch.

In accordance with one or more embodiments, for additional security, in some cases, a two-step authentication process is required at the computing device. In such instances, two different authentication protocols may be implemented which provides the opportunity to acquire two sets of complementary medical data for supply to the digital twin 412. The digital twin processing arrangement 420 (or a different component of the system) may provide an indication of desired combinations of medical data to collect concurrently in this way. For example, if a goal is to determine exercise tolerance for a person, after a period of high activity has been recognized, the authentication settings of the computing device may be set to utilize facial recognition, to allow determination of heart rate, followed by speech recognition, wherein the user is prompted for example to answer a question about their current fatigue level.

Embodiments of the invention described above employ a processing arrangement. The processing arrangement may in general comprise a single processor or a plurality of processors. It may be located in a single containing device, structure or unit, or it may be distributed between a plurality of different devices, structures or units. Reference therefore to the processing arrangement being adapted or configured to perform a particular step or task may correspond to that step or task being performed by any one or more of a plurality of processing components, either alone or in combination. The skilled person will understand how such a distributed processing arrangement can be implemented.

The one or more processors of the processing arrangement can be implemented in numerous ways, with software and/or hardware, to perform the various functions required. A processor typically employs one or more microprocessors that may be programmed using software (e.g., microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

A single processor or other unit may fulfill the functions of several items recited in the claims.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”.

Any reference signs in the claims should not be construed as limiting the scope.

1. A computer-implemented method for configuring settings of a biometric authentication function of a computing device based on input data requirements of a personalized digital model of at least a portion of an anatomy of a patient, the method comprising:
obtaining a first input indicative of a set of input medical data requirements of the digital model for obtaining, based on running simulations on the digital model, a desired set of output information from the model,
obtaining a second input indicative of one or more biometric authentication protocols being executable by the computing device for performing the biometric authentication function on the computing device, wherein each authentication protocol is associated with a set of biometric input data requirements;
comparing the input medical data requirements of the digital model with the biometric input data requirements of the one or more authentication protocols;
configuring an authentication protocol setting of the computing device based on the digital model input data requirements and based on said comparison.

2. A method as claimed in claim 1, wherein the method comprises obtaining an indication of a scheduled authentication event comprising scheduled implementation of an authentication protocol, or comprises obtaining an indication of a default authentication protocol setting of the computing device, and wherein the configuring comprises altering the scheduled or default authentication protocol, or settings thereof.

3. A method as claimed in claim 1, wherein the second input is indicative of a set of multiple biometric authentication protocols being selectively executable by the computing device, and wherein the configuring the authentication protocol setting comprises selecting one of the authentication protocols.

4. A method as claimed in claim 1, wherein the first and second inputs include, or the method comprises determining, required sensing data to be obtained by the computing device to provide, or to be used in providing, the digital model input data and the biometric data, and the comparing comprises comparing the sensing data requirements.

5. A method as claimed in claim 4, wherein the sensing data requirements include at least one suitable sensing modality, and optionally suitable ranges for one or more sensing modality acquisition parameters.

6. A method as claimed in claim 5, wherein configuring the authentication protocol setting includes: selecting one of the one or more authentication protocols and/or configuring one or more sensing modality acquisition parameters.

7. A method as claimed in claim 4, wherein the authentication protocol setting is configured so as to achieve at least a partial match between sensing data requirements of an authentication protocol implemented according to the configured setting and those of the medical input data requirements.

8. A method as claimed in claim 1, wherein the configuring an authentication protocol setting comprises issuing a control instruction to cause the computing device to implement the authentication protocol setting on at least one future authentication event.

9. A method as claimed in claim 1, wherein the computing device is a mobile computing device.

10. A method as claimed in claim 1, wherein the first input includes an indication of one or more sets of predicted future data input requirements of the model at one or more future times, and wherein configuring the authentication protocol setting comprises issuing a control instruction to cause the computing device to schedule implementation of the authentication protocol setting at the one or more relevant future times.

11. A computer program product comprising computer program code, the computer program code being executable on a processor or computer to cause the processor or computer to perform a method in accordance with claim 1.

12. A system for use in configuring settings of a biometric authentication function of a computing device based on input data requirements of a personalized digital model of at least a portion of an anatomy of a patient, the system comprising:
a primary processing arrangement having an input/output for receiving and outputting data, and
the primary processing arrangement adapted to:
receive a first input indicative of a set of input medical data requirements of the digital model for obtaining, based on running simulations on the digital model, a desired set of output information from the model,
receive a second input indicative of one or more biometric authentication protocols being executable by the computing device for performing the biometric authentication function on the computing device, wherein each authentication protocol is associated with a set of biometric input data requirements;
perform a comparison procedure between the input medical data requirements of the digital model and the biometric input data requirements of the one or more authentication protocols;
configure an authentication protocol setting of the computing device based on the digital model input data requirements and based on said comparison.

13. A system as claim in claim 12, wherein the system further comprises a digital model section, comprising:
a data storage arrangement, storing a digital model of at least a portion of an anatomy of the patient, configured to receive one or more model inputs and to simulate an actual physical state of said at least part of the anatomy based on the inputs, and for generating one or more model outputs relating to a current or future state of the anatomy;

14. A system as claimed in claim 12, wherein the system includes a further processing arrangement for coupling in use to a personalized digital model, and adapted to determine the input data requirements for the digital model based at least in part on a latest set of model outputs from the model, and wherein the primary processing arrangement receives the first input from the further processing arrangement.

15. A system as claimed in claim 12, wherein the system includes the computing device, the computing device is a portable computing device, and wherein the primary processing arrangement is integrated in the portable computing device.
